(19) United States
(12) Patent Application Publication
(10) Pub. No.: US 2026/0318917 A1
(43) Pub. Date: Oct. 15, 2026

(54) BIOMARKERS FOR TUBERCULOSIS AND USES THEREOF

(71) Applicant: UNIVERSITY OF SOUTHAMPTON, Southampton (GB)

(72) Inventors: Paul ELKINGTON, Southampton (GB); Hannah SCHIFF, Southampton (GB); Diana Garay BAQUERO, Southampton (GB)

(21) Appl. No.: 19/483,009

(22) PCT Filed: May 10, 2024
(86) PCT No.: PCT/GB2024/051230

§ 371 Date: Nov. 10, 2025

(30) Foreign Application Priority Data
May 10, 2023 (GB) 2306925.5

(51) Int. Cl. G01N 33/569

(52) U.S. Cl. CPC G01N 33/5695; G01N 2333/35

(57) ABSTRACT

A method of diagnosing tuberculosis in a subject, the method comprising: a) providing a sample obtained from the subject; b) determining the level of at least one protein in the sample, wherein the at least one protein is selected from the group consisting of Transcription termination factor 1, Fetuin-B, Heparin cofactor 2, Alpha-1-antichymotrypsin, Gamma-glutamyl hydrolase, Plastin-2, low affinity immunoglobulin gamma Fc region receptor III-B, and variants thereof; c) comparing the level of the at least one protein from step b) with a reference level of the at least one protein; and d) using the results from c) to diagnose or determine if the subject has tuberculosis, wherein the reference level of the at least one protein is the level of the at least one protein that is present in an individual that does not have tuberculosis, and wherein the subject is identified as having tuberculosis if the level of the at least one protein from step b) is higher than the reference level of the at least one protein.

The present invention relates to novel biomarkers for use in the diagnosis and treatment of tuberculosis.

Tuberculosis (TB) is an infectious disease caused by the bacterium Mycobacterium tuberculosis. TB generally affects the lungs but can also affect other parts of the body. Most infections do not have symptoms, in which case it is known as latent TB.

TB is a globally important infection which continues to cause approximately 10 million cases and 1.5 million deaths per year worldwide. Most cases are in resource poor nations, and it is estimated that 4 million active TB cases are simply not diagnosed each year, and the COVID-19 pandemic has further reduced diagnosis. The diagnosis of TB remains a challenge. There are no specific tests to identify patients who have active pulmonary TB that fulfil all the criteria recommended by the World Health Organisation for an optimal diagnostic assay, and certainly none which can be rolled out widely to screen populations.

Undiagnosed patients are a major reservoir for spread of disease including drug resistant TB. Microbiological techniques required for specific identification and drug susceptibility can take days to weeks and are often not available in resource poor and remote areas. A rapid, accurate, and inexpensive TB test used by personnel in the clinic or local hospital would add tremendous value to public health in areas with limited resources by identifying those in need of treatment rapidly and hence decrease the spread of disease to others. The lack of a point-of-care test has been identified as a major gap in the existing pipeline of TB diagnostics.

The current diagnostic tests rely on acid-fast staining of sputum and chest x-rays, both of which require laboratory space, training and expertise. Furthermore, sputum smear testing has very poor sensitivity and specificity. Therefore, TB diagnosis requires a hospital setting and is frequently unavailable to those who have disease. An ideal TB diagnostic would be one which does not require expensive equipment, can be performed by someone with minimal training and is able to diagnose patients with active pulmonary disease who transmit infection to others.

In the light of the failings of the current tests for TB, there is a need for an improved test, preferably a rapid but highly accurate test. An aim of the present invention is therefore to provide novel biomarkers which can be used in the diagnosis and treatment of TB.

### SUMMARY OF THE INVENTION

Accordingly, in a first aspect of the invention, there is provided a method of diagnosing tuberculosis in a subject, the method comprising:
a) providing a sample obtained from the subject;
b) determining the level of at least one protein in the sample, wherein the at least one protein is selected from the group consisting of Fetuin-B, Heparin cofactor 2, Alpha-1-antichymotrypsin, Gamma-glutamyl hydrolase, Transcription termination factor 1, Plastin-2, low affinity immunoglobulin gamma Fc region receptor III-B, and variants thereof;
c) comparing the level of the at least one protein from step b) with a reference level of the at least one protein; and
d) using the results from c) to diagnose or determine if the subject has tuberculosis,
wherein the reference level of the at least one protein is the level of the at least one protein that is present in an individual that does not have tuberculosis, and
wherein the subject is identified as having tuberculosis if the level of the at least one protein from step b) is higher than the reference level of the at least one protein.

A “variant” may be understood by the skilled person to include i) a functional variant of fragment of a claimed sequence, ii) a variant or fragment of a claimed sequence which includes conservative amino acid substitutions, or iii) a variant or fragment of a claimed sequence which has at least 70%, 75%, 80%, 85%, 90%, 95%, 98%, 99% or more sequence identity with the claimed sequence. A fragment may include a protein comprising or consisting of a sequence of at least 50%, 60%, 70%, 75%, 80%, 90%, 95%, 98%, 99% or more of the claimed sequence.

Percentage amino acid sequence identity is defined as the percentage of amino acid residues in a sequence that is identical with the amino acids in the sequence ID numbers referred to after aligning the sequences and introducing gaps if necessary to achieve the maximum percent sequence identity. Alignment for purpose of determining percent sequence identity can be achieved in many ways that are well known to the person skilled in the art, and include, for example, using BLAST and ALIGN algorithms. Sequence identity may be determined by standard BLASTP alignment parameters (provided by http://www.ncbi.nlm.nih.gov/).

In one embodiment of the invention, step b) comprises determining the level of a protein of Transcription termination factor 1 or a variant thereof in the sample. In one embodiment of the invention, step b) does not comprise determining the level of Transcription termination factor 1 or a variant thereof in the sample.

In one embodiment of the invention, step b) comprises determining the level of a protein of Fetuin-B or a variant thereof in the sample.

In one embodiment of the invention, step b) comprises determining the level of Fetuin-B, Heparin cofactor 2, Alpha-1-antichymotrypsin, Gamma-glutamyl hydrolase, Plastin-2, and variants thereof in the sample.

In a preferred embodiment, the tuberculosis is caused by the bacterium Mycobacterium tuberculosis.

In one embodiment of the invention, step b) further comprises determining the level of at least one other protein biomarker of TB in the sample, which will be known by the person skilled in the art. In another embodiment of the invention, step b) further comprises determining the level of at least one other protein biomarker of TB in the sample from Table 1 below. For example, the invention may comprise in step b) determining the level of at least one protein in the sample selected from the group consisting of Low affinity immunoglobulin gamma Fc region receptor III-B, Adenosine deaminase 2, Monocyte differentiation antigen, Leucine-rich alpha-2-glycoprotein, L-selectin and variants thereof. Thus, in one embodiment, the method comprises in step b) determining the level of at least one protein in the sample selected from the group consisting of Fetuin-B, Heparin cofactor 2, Alpha-1-antichymotrypsin, Gamma-glutamyl hydrolase, Transcription termination factor 1, Plastin-2 and variants thereof and determining the level of at least one protein in the sample selected from the group consisting of Low affinity immunoglobulin gamma Fc region receptor III-B, Adenosine deaminase 2, Monocyte differentiation antigen, Leucine-rich alpha-2-glycoprotein, L-selectin and variants thereof.

TABLE 1

| Uniprot | Gene | Protein names |
|---|---|---|
| P02741 | CRP | C-reactive protein |
| P05109 | S100A8 | Protein S100-A8 (Calgranulin-A) |
| | | (Calprotectin L1L subunit) |
| P06702 | S100A9 | Protein S100-A9 (Calgranulin-B) |
| | | (Calprotectin L1H subunit) |
| Q08830 | FGL1 | Fibrinogen-like protein 1 |
| P18428 | LBP | Lipopolysaccharide-binding protein |
| P0DJI9 | SAA2 | Serum amyloid A-2 protein |
| P02750 | LRG1 | Leucine-rich alpha-2-glycoprotein |
| P0DJI8 | SAA1 | Serum amyloid A-1 protein (SAA) |
| P00738 | HP | Haptoglobin (Zonulin) |
| P20848 | SERPINA2 | Putative alpha-1-antitrypsin-related protein |
| | | (Serpin A2) |
| P07988 | SFTPB | Pulmonary surfactant-associated protein B |
| Q9BXR6 | CFHR5 | Complement factor H-related protein 5 |
| P01861 | IGHG4 | Immunoglobulin heavy constant gamma 4 |
| O75015 | FCGR3B | Low affinity immunoglobulin gamma Fc region |
| | | receptor III-B (CD antigen CD16b) |
| P02748 | C9 | Complement component C9 |
| P01009 | SERPINA1 | Alpha-1-antitrypsin (Serpin A1) |
| P02763 | ORM1 | Alpha-1-acid glycoprotein 1 |
| P02671 | FGA | Fibrinogen alpha chain |
| P0C0L4 | C4A | Complement C4-A |
| P00739 | HPR | Haptoglobin-related protein |
| Q9Y275 | TNFSF13B | Tumour necrosis factor ligand superfamily |
| | | member 13B (B lymphocyte stimulator) |
| Q10588 | BST1 | Bone marrow stromal antigen 1 (CD antigen |
| | | CD157) (ADP-ribosyl cyclase 2) |
| Q06033 | ITIH3 | Inter-alpha-trypsin inhibitor heavy chain H3 |
| Q9NZK5 | ADA2 | Adenosine deaminase 2 |
| P04275 | VWF | von Willebrand factor |
| P09960 | LTA4H | Leukotriene A-4 hydrolase |
| P18065 | IGFBP2 | Insulin-like growth factor-binding protein 2 |
| P00751 | CFB | Complement factor B |
| P00740 | F9 | Coagulation factor IX (Christmas factor) |
| P14151 | SELL | L-selectin (CD antigen CD62L) |
| P19652 | ORM2 | Orosomucoid-2 (Alpha-1-acid glycoprotein 2) |
| P00450 | CP | Ceruloplasmin |
| Q8NBJ4 | GOLM1 | Golgi membrane protein 1 |
| Q8WU39 | MZB1 | Marginal zone B- and B1-cell-specific protein |
| P08571 | CD14 | Monocyte differentiation antigen CD14 |
| Q15485 | FCN2 | Ficolin-2 (Ficolin-B) (L-ficolin) |
| P08637 | FCGR3A | Low affinity immunoglobulin gamma Fc region |
| | | receptor III-A (CD16a antigen) |
| P24821 | TNC | Tenascin C (Glioma-associated-extracellular |
| | | matrix antigen) |
| P02675 | FGB | Fibrinogen beta chain |
| P02679 | FGG | Fibrinogen gamma chain |
| P14625 | HSP90B1 | Endoplasmin |
| P13598 | ICAM2 | Intercellular adhesion molecule 2 (CD |
| | | antigen CD102) |
| Q14624 | ITIH4 | Inter-alpha-trypsin inhibitor heavy chain H4 |
| P28062 | PSMB8 | Proteasome subunit beta type-8 |
| P24387 | CRHBP | Corticotropin-releasing factor-binding protein |
| P61626 | LYZ | Lysozyme C |
| P0C0L5 | C4B | Complement C4-B |
| Q9UK55 | SERPINA10 | Protein Z-dependent protease inhibitor |
| | | (Serpin A10) |
| P07358 | C8B | Complement component C8 beta chain |
| P06681 | C2 | Complement C2 |
| P00488 | F13A1 | Coagulation factor XIII A chain |
| P19827 | ITIH1 | Inter-alpha-trypsin inhibitor heavy chain H1 |
| P49908 | SELENOP | Selenoprotein P |
| P36955 | SERPINF1 | Pigment epithelium-derived factor (Serpin F1) |
| P43251 | BTD | Biotinidase |
| Q92520 | FAM3C | Interleukin-like EMT inducer |
| Q04756 | HGFAC | Hepatocyte growth factor activator |
| P19823 | ITIH2 | Inter-alpha-trypsin inhibitor heavy chain H2 |
| P22105 | TNXB | Tenascin-X |
| Q16853 | AOC3 | Membrane primary amine oxidase |
| Q96PD5 | PGLYRP2 | Peptidoglycan recognition protein 2 |
| Q76LX8 | ADAMTS13 | von Willebrand factor-cleaving protease |
| P12830 | CDH1 | Cadherin-1 |
| P02768 | ALB | Albumin |
| P08253 | MMP2 | Matrix metalloproteinase-2 |
| O75882 | ATRN | Attractin |
| P02787 | TF | Serotransferrin (Siderophilin) |
| P81605 | DCD | Dermcidin |
| P02655 | APOC2 | Apolipoprotein C-II |
| P51884 | LUM | Lumican |
| P02765 | AHSG | Alpha-2-HS-glycoprotein (Fetuin-A) |
| P02749 | APOH | Apolipoprotein H |
| P05090 | APOD | Apolipoprotein D |
| P06396 | GSN | Gelsolin |
| P24593 | IGFBP5 | Insulin-like growth factor-binding protein 5 |
| O95445 | APOM | Apolipoprotein M |
| P06727 | APOA4 | Apolipoprotein A-IV |
| Q9H8L6 | MMRN2 | Multimerin-2 (EMILIN-3) |
| P35858 | IGFALS | Insulin-like growth factor-binding protein |
| | | complex acid labile subunit |
| P07148 | FABP1 | Fatty acid-binding protein |
| P55290 | CDH13 | Cadherin-13 (Heart Cadherin) |
| Q9NQ38 | SPINK5 | Serine protease inhibitor Kazal-type 5 |
| P17936 | IGFBP3 | Insulin-like growth factor-binding protein 3 |
| Q16819 | MEP1A | Meprin A subunit alpha |
| Q15166 | PON3 | Serum paraoxonase/lactonase 3 |
| P16035 | TIMP2 | Metalloproteinase inhibitor 2 |
| Q9UMX5 | NENF | Neudesin (Cell immortalization-related |
| | | protein 2) |
| P05452 | CLEC3B | Tetranectin (C-type lectin domain family |
| | | 3 member B) |
| P39059 | COL15A1 | Collagen alpha-1(XV) chain |
| P58335 | ANTXR2 | Anthrax toxin receptor 2 |
| P02656 | APOC3 | Apolipoprotein C-III |
| P43652 | AFM | Afamin (Alpha-albumin) |
| P02652 | APOA2 | Apolipoprotein A-II |
| O60888 | CUTA | Acetylcholinesterase-associated protein |
| Q96KN2 | CNDP1 | Carnosine dipeptidase 1 |
| Q14623 | IHH | Indian hedgehog protein |
| P60985 | KRTDAP | Keratinocyte differentiation-associated |
| | | protein |
| P29622 | SERPINA4 | Kallistatin (Kallikrein inhibitor) |
| | | (Serpin A4) |
| P05154 | SERPINA5 | Plasma serine protease inhibitor (Serpin A5) |
| P80108 | GPLD1 | Phosphatidylinositol-glycan-specific |
| | | phospholipase D (Glycoprotein |
| | | phospholipase D) |
| P22466 | GAL | Galanin peptides |
| Q15828 | CST6 | Cystatin-M (Cystatin-6) |
| P05019 | IGF1 | Insulin-like growth factor I (Somatomedin-C) |
| P30990 | NTS | Neurotensin/neuromedin N |
| Q96EE4 | CCDC126 | Coiled-coil domain-containing protein 126 |
| P02766 | TTR | Transthyretin (Prealbumin) |
| P54802 | NAGLU | Alpha-N-acetylglucosaminidase |
| Q07507 | DPT | Dermatopontin (Tyrosine-rich acidic matrix |
| | | protein) (TRAMP) |
| Q13103 | SPP2 | Secreted phosphoprotein 24 |
| | | (Secreted phosphoprotein 2) |
| P12273 | PIP | Prolactin-inducible protein |
| P23280 | CA6 | Carbonic anhydrase 6 |
| P02753 | RBP4 | Retinol-binding protein 4 |

In one embodiment of the invention, step b) comprises determining the level of at least two, at least three, at least four, at least five, at least six, at least seven, at least eight, at least nine or at least ten proteins in the sample. In another embodiment of the invention, step b) comprises determining the level of two, three, four, five, six, seven, eight, nine or ten proteins in the sample. In another embodiment of the invention, step b) comprises determining the level of six proteins in the sample.

In one embodiment, the method may comprise in step b) determining in the sample the level of Fetuin-B or a variant thereof, and at least one, two, three, four or five protein(s) selected from other protein biomarkers of TB (for example at least one, two, three, four or five protein(s) selected from the group consisting of Heparin cofactor 2, Alpha-1-antichymotrypsin, Gamma-glutamyl hydrolase, Transcription termination factor 1, Plastin-2, Low affinity immunoglobulin gamma Fc region receptor III-B, Adenosine deaminase 2, Monocyte differentiation antigen, Leucine-rich alpha-2-glycoprotein, L-selectin and variants thereof). For example, the method may comprise in step b) determining the level of Fetuin-B or a variant thereof, Heparin cofactor 2 or a variant thereof, Alpha-1-antichymotrypsin or a variant thereof and Gamma-glutamyl hydrolase or a variant thereof.

In another embodiment, the method may comprise in step b) determining in the sample the level of Fetuin-B or a variant thereof, Low affinity immunoglobulin gamma Fc region receptor III-B or a variant thereof and at least one, two, three, or four protein(s) selected from other protein biomarkers of TB (for example at least one, two, three, or four protein(s) selected from the group consisting of Heparin cofactor 2, Alpha-1-antichymotrypsin, Gamma-glutamyl hydrolase, Transcription termination factor 1, Plastin-2, Adenosine deaminase 2, Monocyte differentiation antigen, Leucine-rich alpha-2-glycoprotein, L-selectin and variants thereof).

In another embodiment, the method may comprise in step b) determining in the sample the level of Fetuin-B or a variant thereof, Heparin cofactor 2 or a variant thereof, Low affinity immunoglobulin gamma Fc region receptor III-B or a variant thereof and at least one, two or three protein(s) selected from other protein biomarkers of TB (for example at least one, two or three protein(s) selected from the group consisting of Alpha-1-antichymotrypsin, Gamma-glutamyl hydrolase, Transcription termination factor 1, Plastin-2, Adenosine deaminase 2, Monocyte differentiation antigen, Leucine-rich alpha-2-glycoprotein, L-selectin, and variants thereof.

In another embodiment, the method may comprise in step b) determining in the sample the level of Low affinity immunoglobulin gamma Fc region receptor III-B, Fetuin-B, Adenosine deaminase 2, Monocyte differentiation antigen, Leucine-rich alpha-2-glycoprotein, L-selectin, and variants thereof.

In one embodiment, the method may comprise in step b) determining in the sample the level of Transcription termination factor 1 or a variant thereof, and at least one, two, three, four or five protein(s) selected from other protein biomarkers of TB (for example at least one, two, three, four or five protein(s) selected from the group consisting of Fetuin-B, Heparin cofactor 2, Alpha-1-antichymotrypsin, Gamma-glutamyl hydrolase, Plastin-2, Low affinity immunoglobulin gamma Fc region receptor III-B, Adenosine deaminase 2, Monocyte differentiation antigen, Leucine-rich alpha-2-glycoprotein, L-selectin and variants thereof). For example, the method may comprise in step b) determining the level of Transcription termination factor 1 or a variant thereof and Fetuin-B or a variant thereof.

In another embodiment, the method may comprise in step b) determining the level of Transcription termination factor 1 or a variant thereof and Low affinity immunoglobulin gamma Fc region receptor III-B or a variant thereof.

In another embodiment, the method may comprise in step b) determining in the sample the level of Transcription termination factor 1 or a variant thereof, Fetuin-B or a variant thereof and Low affinity immunoglobulin gamma Fc region receptor III-B or a variant thereof.

In another embodiment, the method may comprise in step b) determining in the sample the level of Transcription termination factor 1 or a variant thereof, Fetuin-B or a variant thereof and Low affinity immunoglobulin gamma Fc region receptor III-B or a variant thereof, and at least one, two, three, or four protein(s) selected from other protein biomarkers of TB (for example at least one, two, three, or four protein(s) selected from the group consisting of Heparin cofactor 2, Alpha-1-antichymotrypsin, Gamma-glutamyl hydrolase, Plastin-2, Adenosine deaminase 2, Monocyte differentiation antigen, Leucine-rich alpha-2-glycoprotein, L-selectin and variants thereof).

In another embodiment, the method may comprise in step b) determining in the sample the level of Fetuin-B and Low affinity immunoglobulin gamma Fc region receptor III-B.

In another embodiment, the method may comprise in step b) determining in the sample the level of Fetuin-B or a variant thereof and at least one, two, three, or four protein(s) selected from the group consisting of Low affinity immunoglobulin gamma Fc region receptor III-B, Heparin cofactor 2, Alpha-1-antichymotrypsin, Gamma-glutamyl hydrolase, Plastin-2, Adenosine deaminase 2, Monocyte differentiation antigen, Leucine-rich alpha-2-glycoprotein, L-selectin and variants thereof.

In one embodiment, the level of Transcription termination factor 1 is not determined.

The reference level of a protein is the amount of that protein that is present in an individual that does not have tuberculosis. Preferably, the reference level is obtained from a control subject that does not have tuberculosis.

A subject is identified as having tuberculosis if the level of a protein is higher than the reference level of that protein. When the level of two or more proteins from step b) are higher than their respective reference levels, this may provide a strong indication of tuberculosis infection in the subject. By increasing the number of proteins considered the specificity and sensitivity of the method may be increased.

A subject may be identified as having tuberculosis if the level of the at least one protein from step b) is at least about 20% higher, at least about 25% higher, at least about 30% higher, at least about 33% higher, at least about 35% higher, at least about 40% higher, at least about 45% higher, at least about 50% higher, at least about 55% higher about, at least about 60% higher, at least about 65% higher, at least about 70% higher, at least about 75% higher, at least about 80% higher, at least about 90% higher, at least about 95% higher, or at least about 100% higher, than the reference level of that protein. In a preferred embodiment of the invention, a subject may be identified as having tuberculosis if the level of the at least one protein from step b) is at least about 30% higher than the reference level of that protein.

A subject may be identified as having tuberculosis if the level of Fetuin-B or variant thereof from step b) is at least about 40% higher, at least about 45% higher or at least about 50% higher than the reference level of Fetuin-B. A subject may be identified as having tuberculosis if the level of Heparin cofactor 2 or variant thereof from step b) is at least about 20% higher, at least about 25% higher or at least about 30% higher than the reference level of Heparin cofactor 2. A subject may be identified as having tuberculosis if the level of a protein of Alpha-1-antichymotrypsin or variant thereof from step b) is at least about 30% higher, at least about 35% higher, at least about 40% higher than the reference level of Alpha-1-antichymotrypsin. A subject may be identified as having tuberculosis if the level of a protein of Gamma-glutamyl hydrolase or variant thereof from step b) is at least about 40% higher, at least about 45% higher or at least about 50% higher than the reference level of Gamma-glutamyl hydrolase. A subject may be identified as having tuberculosis if the level of a protein of Transcription termination factor 1 or variant thereof from step b) is at least about 90% higher, at least about 95% higher or at least about 100% higher than the reference level of Transcription termination factor 1. A subject may be identified as having tuberculosis if the level of a protein of Plastin-2 or variant thereof from step b) is at least about 70% higher, at least about 75% higher or at least about 80% higher than the reference level of Plastin-2. A subject may be identified as having tuberculosis if the level of a protein of low affinity immunoglobulin gamma Fc region receptor III-B or variant thereof from step b) is at least about 70% higher, at least about 75% higher or at least about 80% higher than the reference level of low affinity immunoglobulin gamma Fc region receptor III-B.

The presence and the level of a protein or a variant thereof in a sample may be determined by any suitable assay, which may comprise the use of any of the group selected from immunoassays, spectrometry, western blot, ELISA, immunoprecipitation, slot or dot blot assay, isoelectric focussing, SDS-PAGE and antibody microarray, immunohistological staining, radio immuno assay (RIA), fluoroimmunoassay, an immunoassay using an avidin-biotin or streptoavidin-biotin system, etc and combinations thereof. In an embodiment the assay may be provided as point of case test, for example, it may use a lateral flow immunoassay. These methods are well known to persons skilled in the art.

The sample may be a sample of any bodily fluid, the bodily fluid may be blood, serum, plasma, urine, sputum, pleural fluid, ascites, lymph node aspirate, bronchial lavage, gastric aspirate, or a stool sample derived from the subject.

The subject may be a mammal, and is preferably a human, but may alternatively be a cow, badger, chicken, elephant, monkey, ape, cat, dog, cow, horse, badger, rabbit or rodent. In a preferred embodiment the method of the invention is used to diagnose tuberculosis in humans, preferably where the disease is caused by Mycobacterium tuberculosis.

The method may further comprise relaying information regarding the tuberculosis diagnosis of a subject to a third party, such as a doctor, other medical professional, pharmacist or other interested party. This information may be relayed digitally, for example via email, SMS or other digital means. This information may be used to determine the appropriate treatment for a subject having tuberculosis. For example, for subjects identified as having high levels of step b) protein(s), treatment may be initiated or additional tests such as chest X-rays and sputum analysis may be requested. Reference to high levels of protein in this context may refer to a level within the top 30% of levels observed in a cohort of patients with tuberculosis who may then require intensified treatment. The cohort may comprise 50 patients or more.

In another aspect of the invention, there is provided a method of treating tuberculosis in a subject, wherein the method comprises:
a) providing a sample obtained from the subject;
b) determining the level of at least one protein in the sample, wherein the at least one protein is selected from the group consisting of Fetuin-B, Heparin cofactor 2, Alpha-1-antichymotrypsin, Gamma-glutamyl hydrolase, Transcription termination factor 1, Plastin-2, low affinity immunoglobulin gamma Fc region receptor III-B, and variants thereof;
c) comparing the level of the at least one protein from step b) with a reference level of the at least one protein; and
d) administering a tuberculosis therapy to the subject if the level of the at least one protein from step b) is higher than the reference level of the at least one protein,
wherein the reference level of the at least one protein is the level of the at least one protein that is present in an individual that does not have tuberculosis.

In one embodiment, the method may further comprise carrying out an additional tuberculosis diagnostic test after step c) to further confirm that the subject has tuberculosis before administering a tuberculosis therapy in step d).

In one embodiment, the tuberculosis therapy is antibiotics. The standard tuberculosis treatment is a 6-month course of antibiotics, with four antibiotics for 2 months, then two antibiotics for the following 4 months. Antibiotics that may be used include isoniazid, rifampicin, pyrazinamide and ethambutol. By using the present method to diagnose patients, unnecessary and/or prolonged antibiotic use may be reduced.

In another aspect of the invention, there is provided a method of selecting a subject for treatment for tuberculosis comprising:
a) providing a sample obtained from the subject;
b) determining the level of at least one protein in the sample, wherein the at least one protein is selected from the group consisting of Fetuin-B, Heparin cofactor 2, Alpha-1-antichymotrypsin, Gamma-glutamyl hydrolase, Transcription termination factor 1, Plastin-2, low affinity immunoglobulin gamma Fc region receptor III-B, and variants thereof;
c) comparing the level of the at least one protein from step b) with a reference level of the at least one protein; and
d) selecting the subject for treatment if the level of the at least one protein from step b) is higher than the reference level of the at least one protein, wherein the reference level of the at least one protein is the level of the at least one protein that is present in an individual that does not have tuberculosis.

In one embodiment, the method may further comprise carrying out an additional tuberculosis diagnostic test after step c) to further confirm that the subject has tuberculosis before selecting the subject for treatment in step d).

In another aspect of the invention, there is provided a kit comprising at least one agent for detecting the level of at least one protein selected from the group consisting of Transcription termination factor 1, Fetuin-B, Heparin cofactor 2, Alpha-1-antichymotrypsin, Gamma-glutamyl hydrolase, Plastin-2, low affinity immunoglobulin gamma Fc region receptor III-B, and variants thereof. The kit may comprise agents for detecting the level of at least two, three, four, five, six, seven or more proteins selected from the group consisting of Transcription termination factor 1, Fetuin-B, Heparin cofactor 2, Alpha-1-antichymotrypsin, Gamma-glutamyl hydrolase, Plastin-2, low affinity immunoglobulin gamma Fc region receptor III-B, and variants thereof. The kit may comprise instructions to use the kit. The kit may be for use with a sample provided by a subject.

In another aspect of the invention, there is provided a kit for use in diagnosing tuberculosis in a subject comprising at least one agent for detecting the level of at least one protein selected from the group consisting of Fetuin-B, Heparin cofactor 2, Alpha-1-antichymotrypsin, Gamma-glutamyl hydrolase, Transcription termination factor 1, Plastin-2, low affinity immunoglobulin gamma Fc region receptor III-B, and variants thereof.

The kit may comprise instructions for suitable operational parameters in the form of a label or separate insert. The instructions may inform a user about how to collect the sample. The instructions may inform a user how to use the kit to diagnose tuberculosis.

In another aspect of the invention, there is provided a composition comprising at least one agent for detecting the level of at least one protein selected from the group consisting of Transcription termination factor 1, Fetuin-B, Heparin cofactor 2, Alpha-1-antichymotrypsin, Gamma-glutamyl hydrolase, Plastin-2, low affinity immunoglobulin gamma Fc region receptor III-B, and variants thereof. The composition may comprise agents for detecting the level of at least two, three, four, five, six, seven or more proteins selected from the group consisting of Transcription termination factor 1, Fetuin-B, Heparin cofactor 2, Alpha-1-antichymotrypsin, Gamma-glutamyl hydrolase, Plastin-2, low affinity immunoglobulin gamma Fc region receptor III-B, and variants thereof.

In another aspect of the invention, there is provided the use of one or more reagents which detect one or more protein selected from the group consisting of Transcription termination factor 1, Fetuin-B, Heparin cofactor 2, Alpha-1-antichymotrypsin, Gamma-glutamyl hydrolase, Plastin-2, low affinity immunoglobulin gamma Fc region receptor III-B, and variants thereof, in the preparation of an agent or composition for determining the tuberculosis disease status of a subject. Determining the tuberculosis disease status of a subject may include determining is a subject does or does not have tuberculosis.

The agent in any aspect or embodiment of the invention may be an antibody.

The person skilled in the art will appreciate that features of any one embodiment and/or aspect of the invention may be applied to all other embodiments and/or aspects of the invention.

Examples embodying an aspect of the invention will now be described with reference to the following figures:

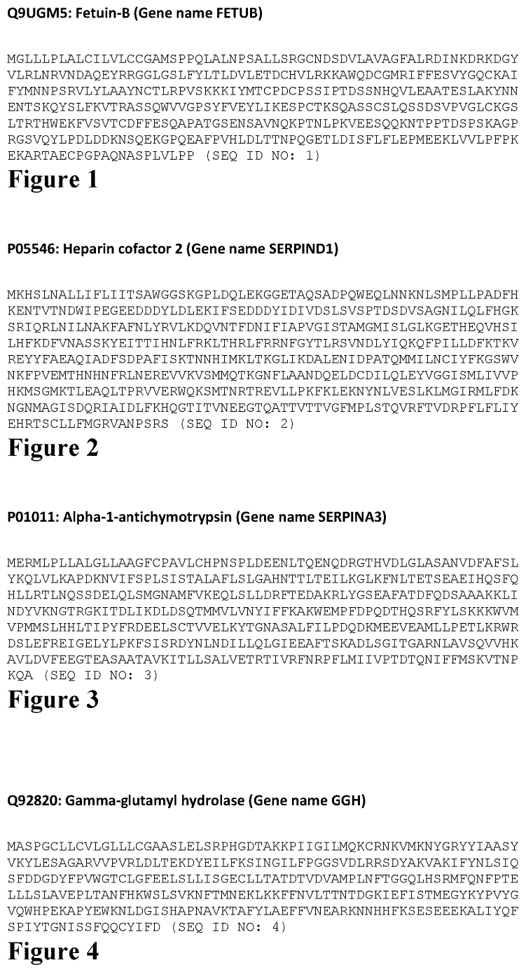

FIG. 1—is SEQ ID NO: 1—the sequence listing of Fetuin-B (Gene name FETUB) having UniProt accession number Q9UGM5.

FIG. 2—is SEQ ID NO: 2—the sequence listing of Heparin cofactor 2 (Gene name SERPIND1) having UniProt accession number P05546.

FIG. 3—is SEQ ID NO: 3—the sequence listing of Alpha-1-antichymotrypsin (Gene name SERPINA3) having UniProt accession number P01011.

FIG. 4—is SEQ ID NO: 4—the sequence listing of Gamma-glutamyl hydrolase (Gene name GGH) having UniProt accession number Q92820.

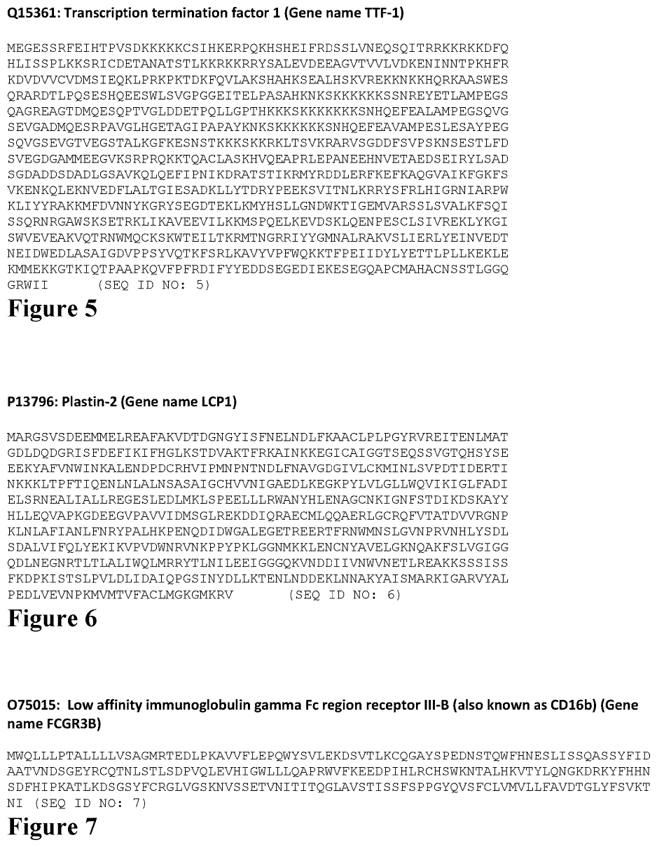

FIG. 5—is SEQ ID NO: 5—the sequence listing of Transcription termination factor 1 (Gene name TTF-1) having UniProt accession number Q15361.

FIG. 6—is SEQ ID NO: 6—the sequence listing of Plastin-2 (Gene name LCP1) having UniProt accession number P13796.

FIG. 7—is SEQ ID NO: 7—the sequence listing of Low affinity immunoglobulin gamma Fc region receptor III-B (also known as CD16b) (Gene name FCGR3B) having UniProt accession number 075015.

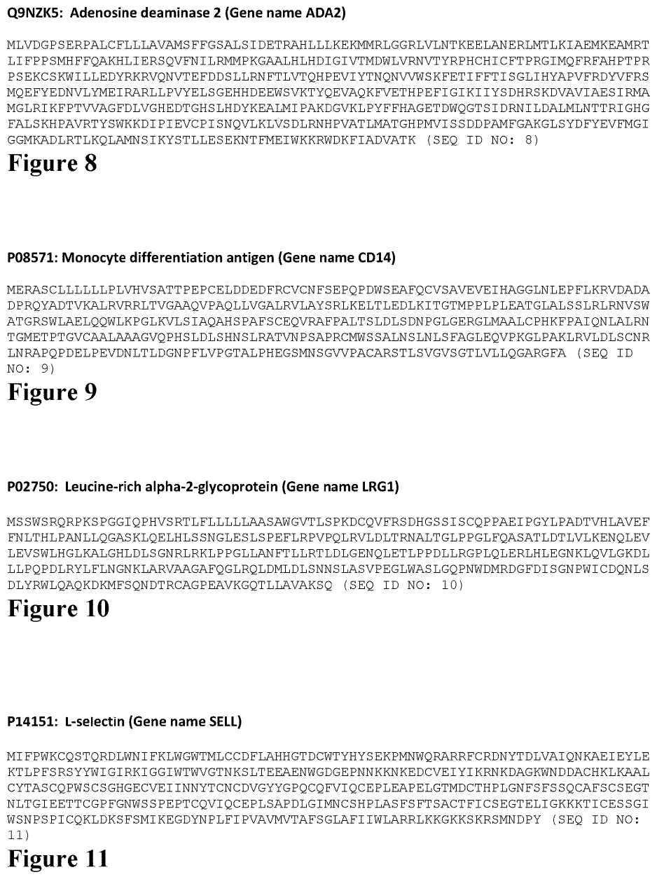

FIG. 8—is SEQ ID NO: 8—the sequence listing of Adenosine deaminase 2 (Gene name ADA2) having UniProt accession number Q9NZK5.

FIG. 9—is SEQ ID NO: 9—the sequence listing of Monocyte differentiation antigen (Gene name CD14) having UniProt accession number P08571.

FIG. 10—is SEQ ID NO: 10—the sequence listing of Leucine-rich alpha-2-glycoprotein (Gene name LRG1) having UniProt accession number P02750.

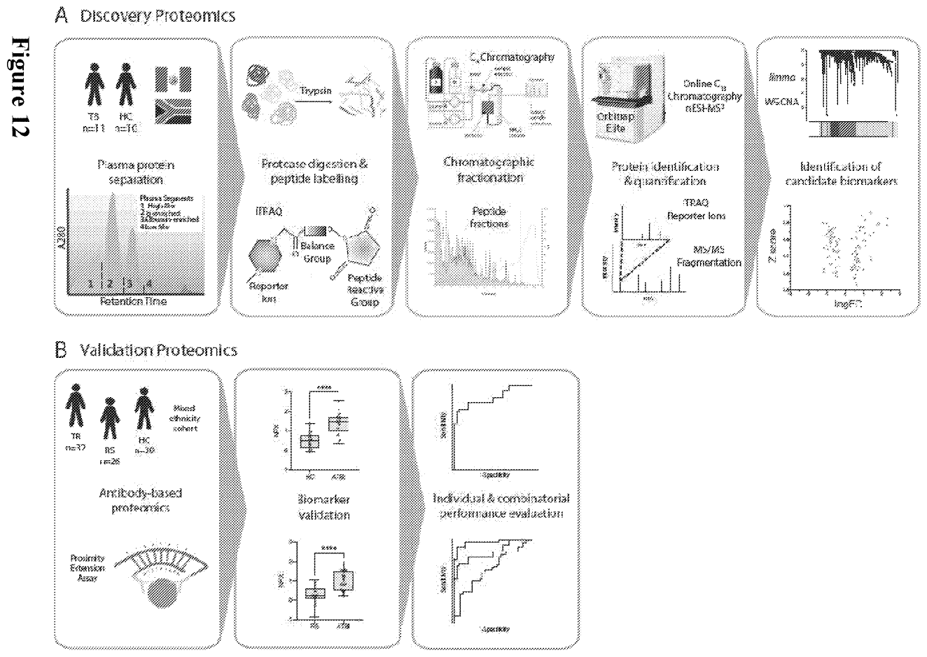

FIG. 11—is SEQ ID NO: 11—the sequence listing of L-selectin (Gene name SELL) having UniProt accession number P14151.

FIG. 12—gives a schematic overview of the discovery and validation proteomic workflow.

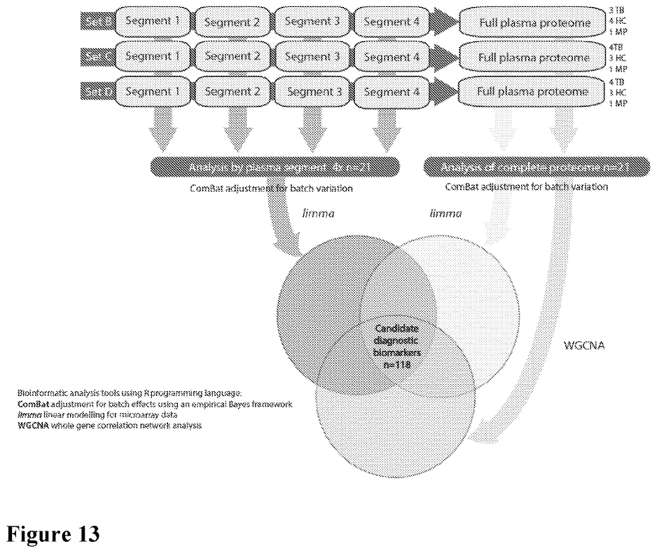

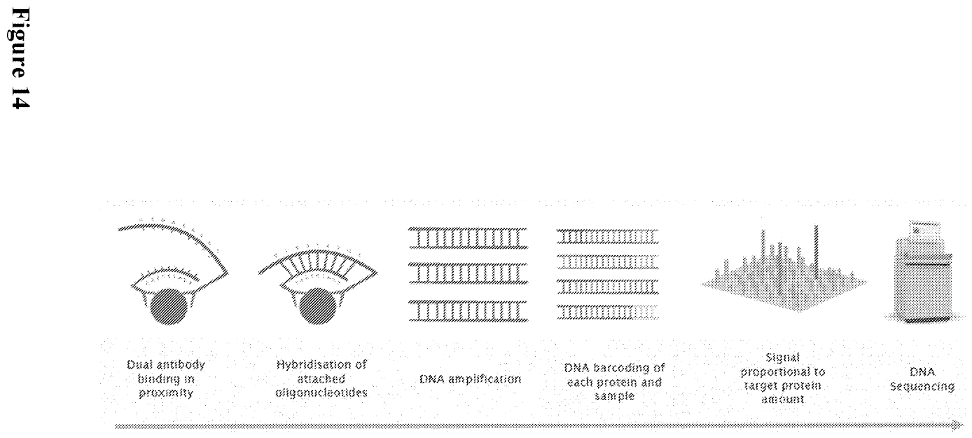

FIG. 13—illustrates the bioinformatic data analysis pipelines from the discovery proteomics to identify differentially expressed proteins.

FIG. 14—gives an overview of the validation proteomics methodology.

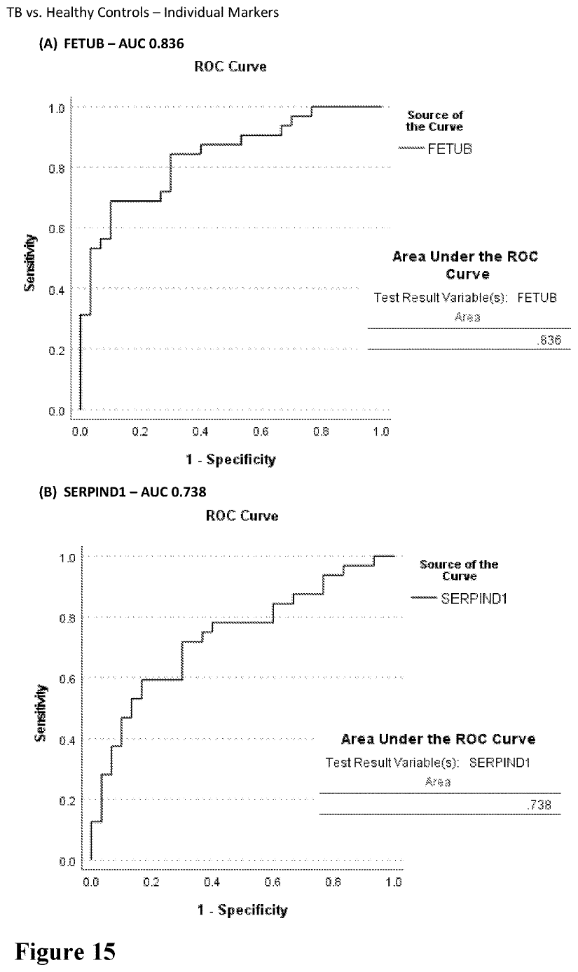

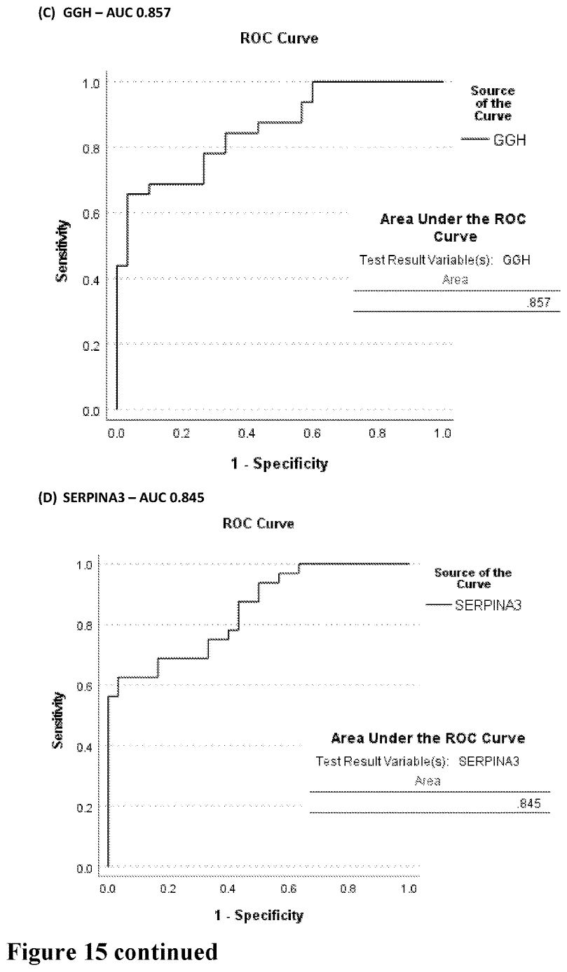

FIG. 15—shows the receiver operating characteristic curves for individual markers validated by proximity extension assay when classifying pulmonary tuberculosis from healthy controls.

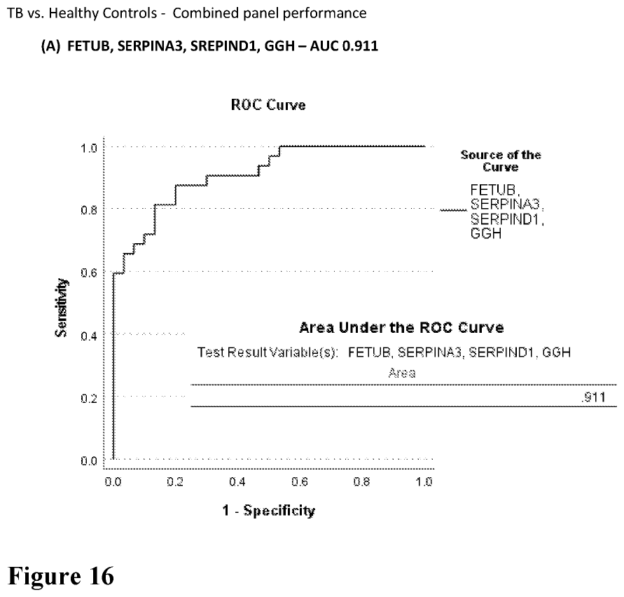

FIG. 16—illustrates that proteins in combination outperform individual marker performance when classifying pulmonary tuberculosis from healthy controls.

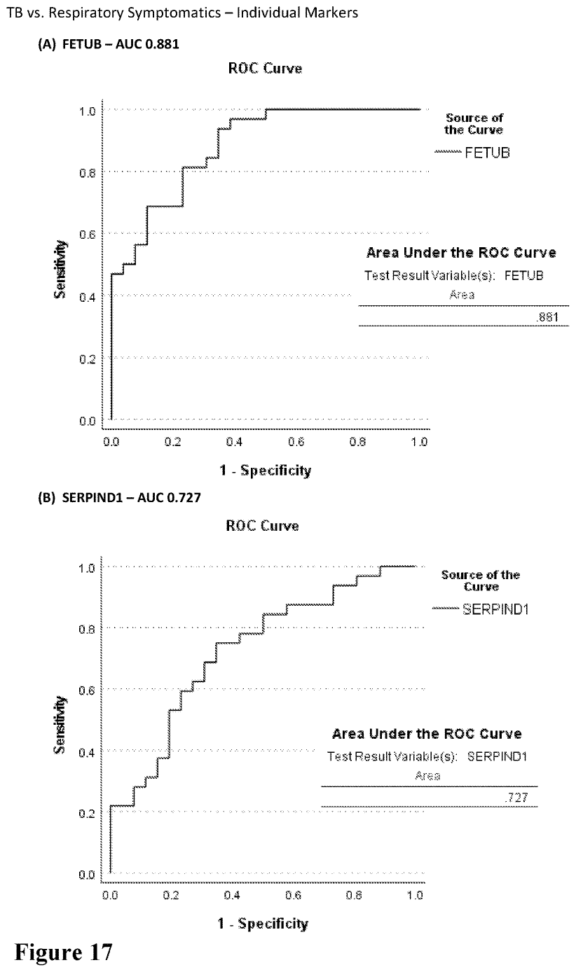

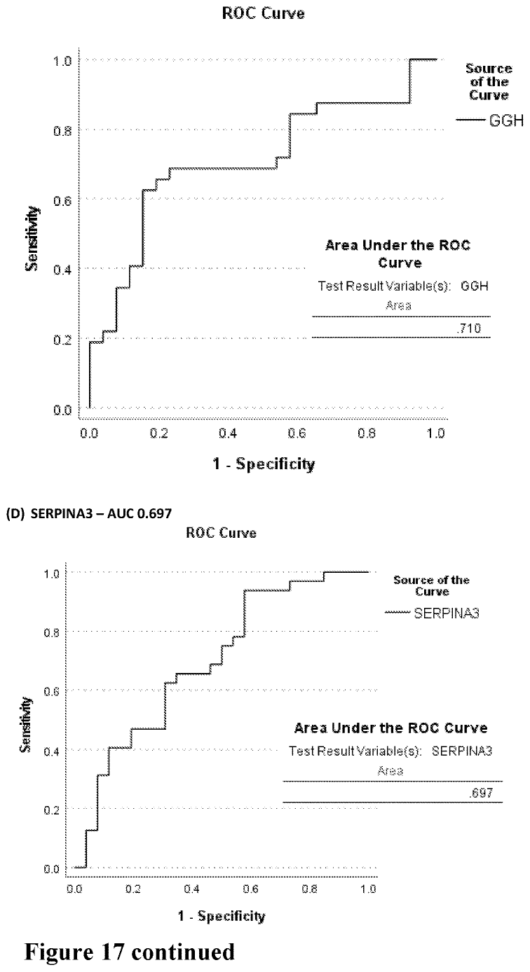

FIG. 17—shows the receiver operating characteristic curves for individual markers validated by proximity extension assay when classifying pulmonary tuberculosis from respiratory symptomatics.

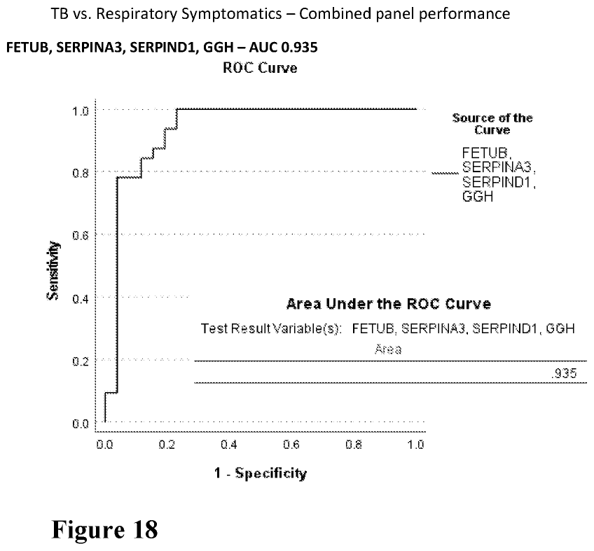

FIG. 18—illustrates that proteins in combination outperform individual marker performance when classifying pulmonary tuberculosis from respiratory symptomatics.

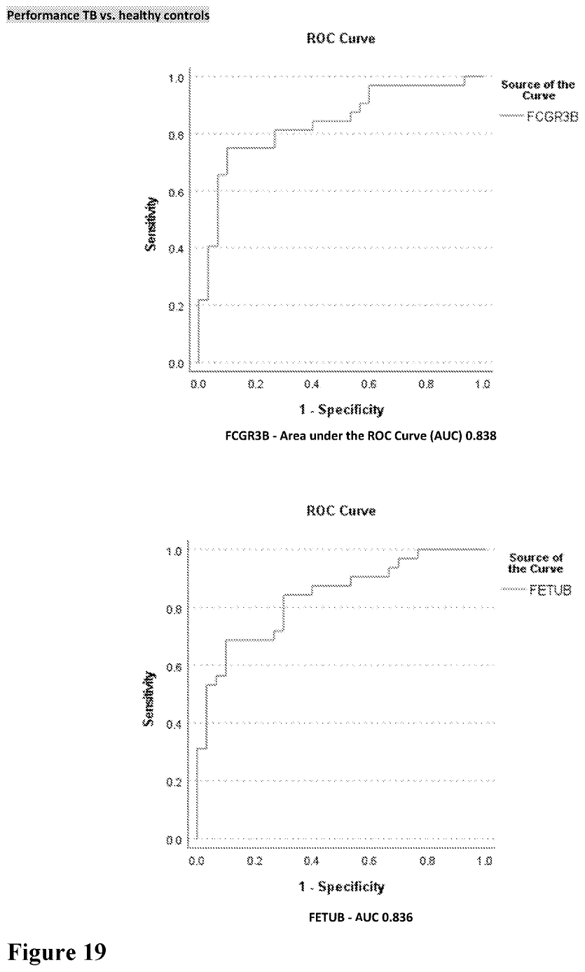

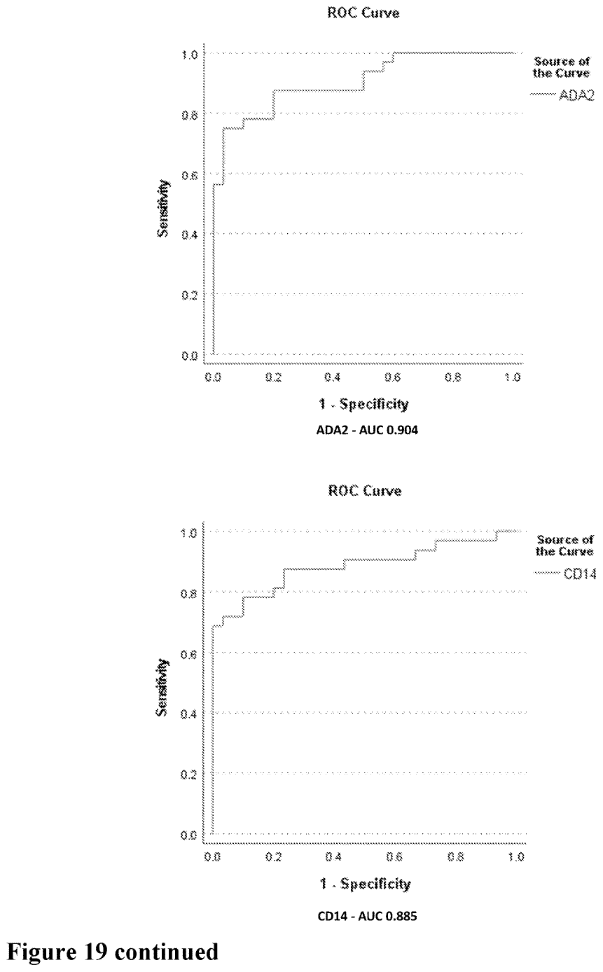

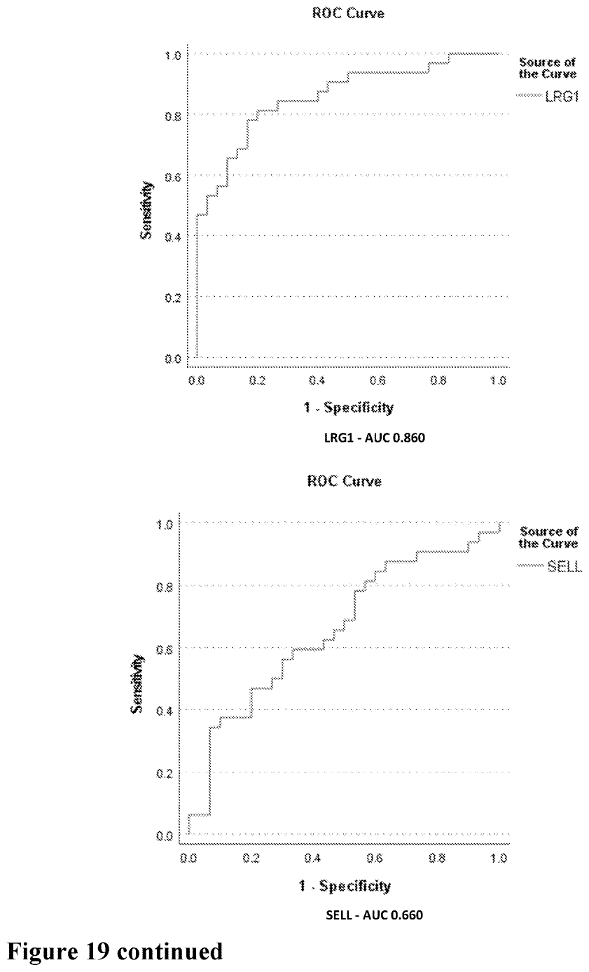

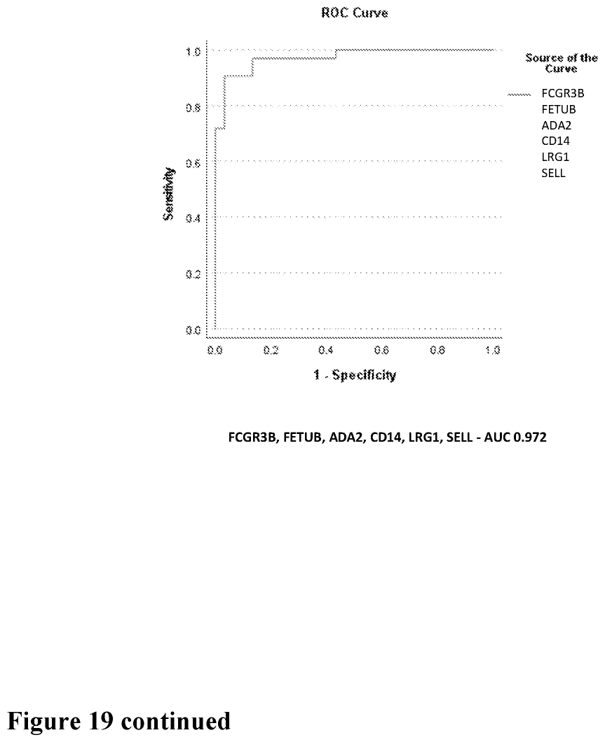

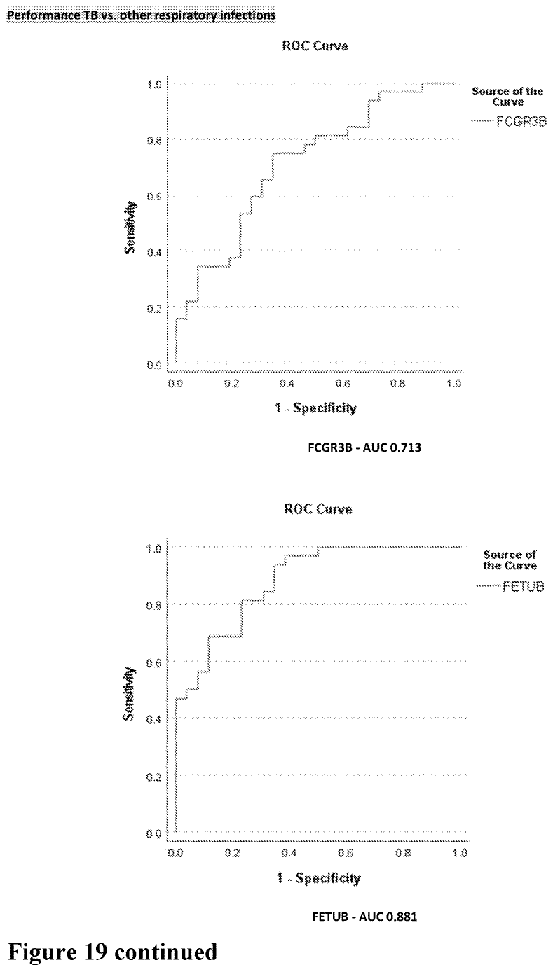

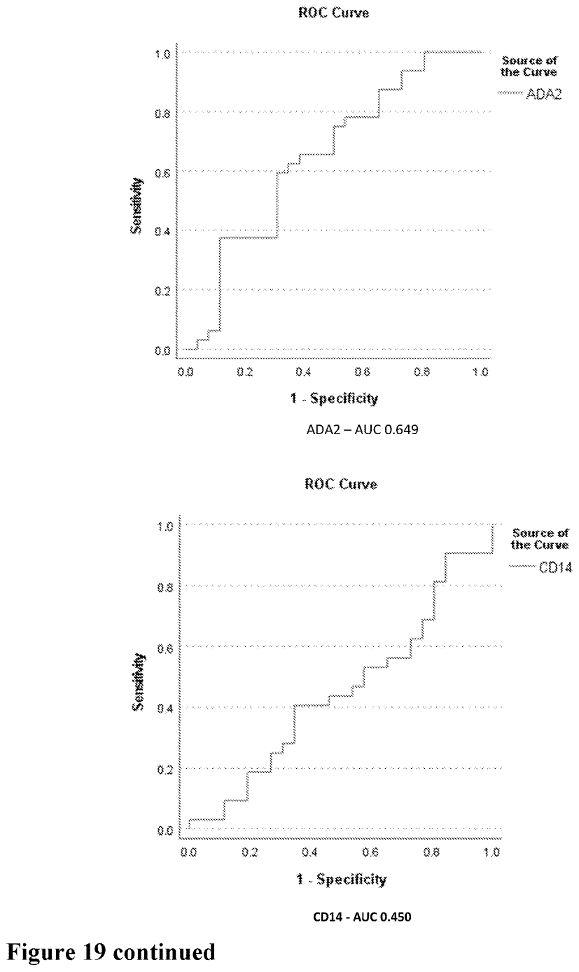

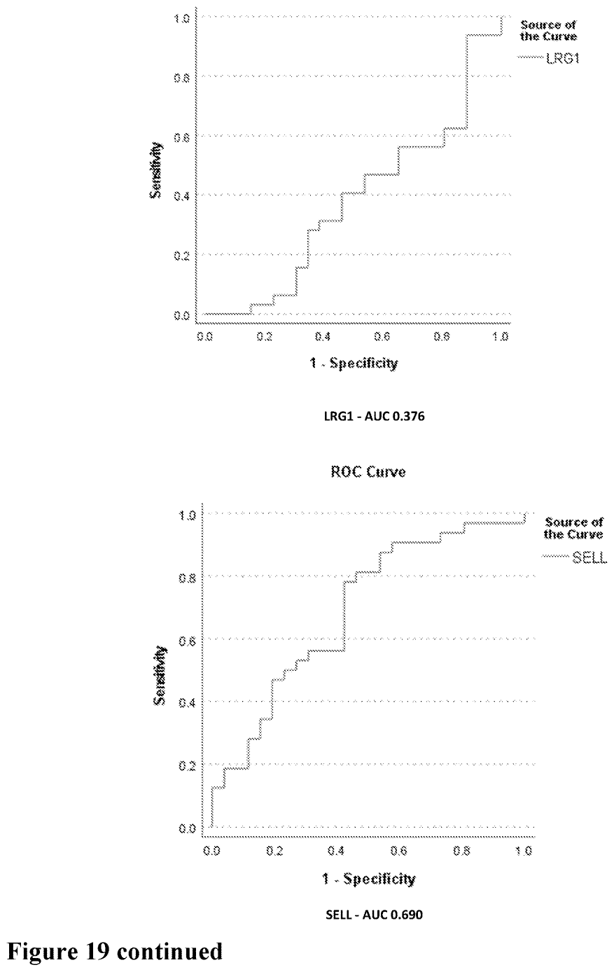

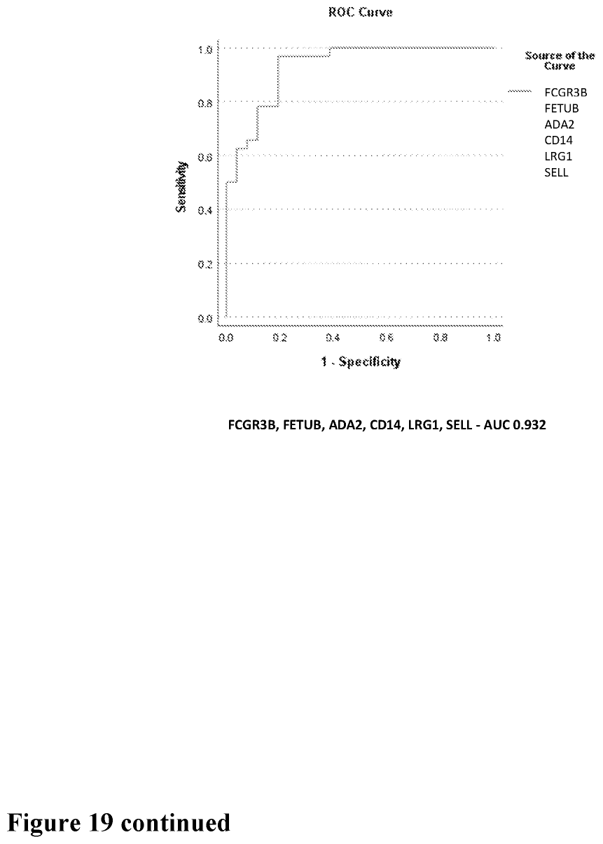

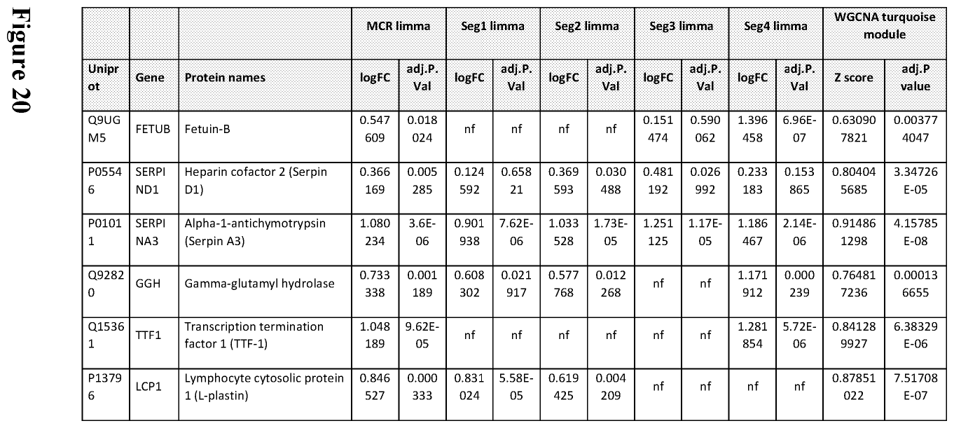

FIG. 19—shows the receiver operating characteristic curves for individual markers validated by proximity extension assay when classifying pulmonary tuberculosis from healthy controls (left-hand column) and respiratory symptomatics (right-hand column) and also illustrates that these proteins in combination outperform their individual marker performance.

FIG. 20—shows the log fold changes of the novel protein biomarkers identified from the discovery proteomic methodology and relevant metrics from the bioinformatic analysis, such as correlation score to pulmonary tuberculosis by whole gene correlation network analysis.

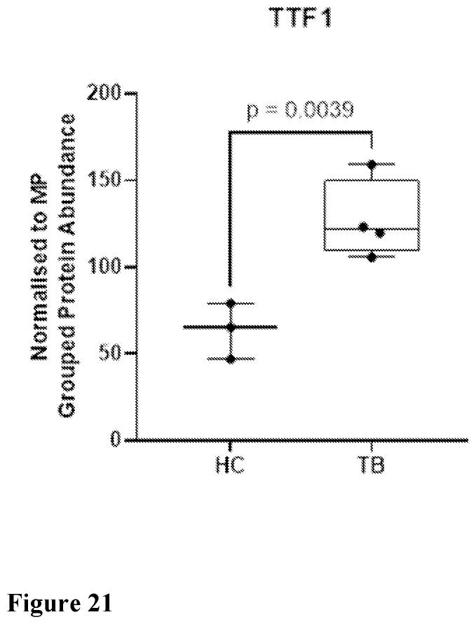

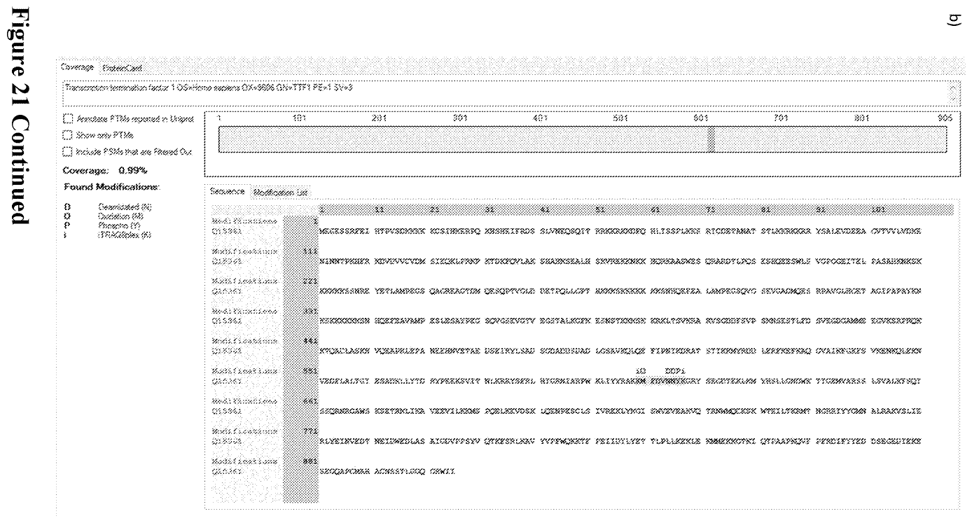

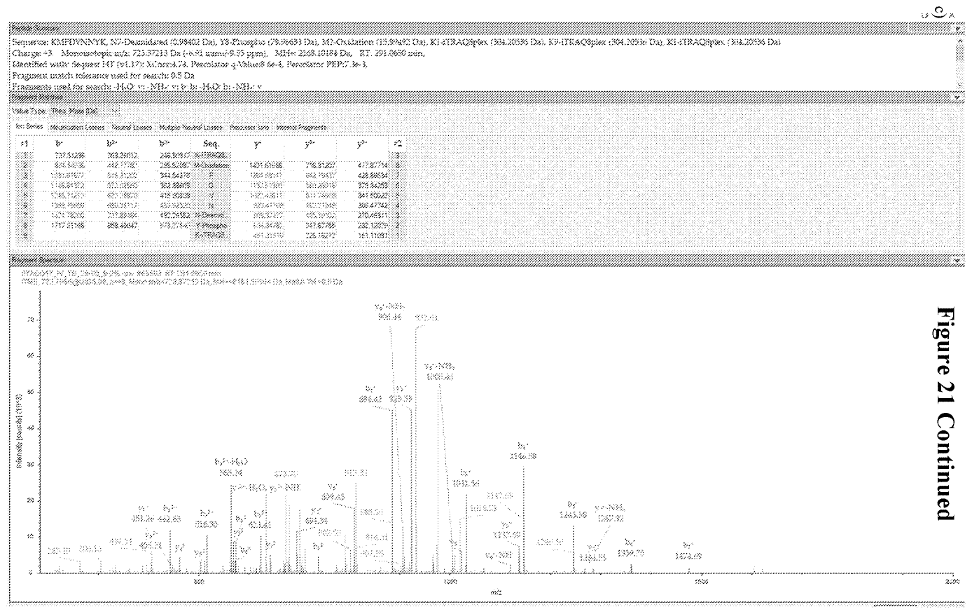

FIG. 21—a) show the significantly elevated abundance of TTF1 from discovery mass spectrometry proteomics in TB compared to healthy controls; b) shows the sequence of TTF-1 showing identified peptide sequence; c) shows an example mass spectrum of the TTF1 peptide identified.

### METHODOLOGICAL DETAILS

### Discovery Proteomics

Twenty-one samples, eleven active pulmonary patients and ten healthy control individuals from African and South American ethnicities were used to profile the active pulmonary tuberculosis plasma proteome. Additionally, a master pool was included for controlling variability across experiments. An aliquot of 20 μL of seven samples from each group South African control, Peruvian control, South African active tuberculosis and Peruvian active pulmonary, were pooled together to prepare the masterpool and aliquoted to prevent freeze-thaw cycles. Allocation of labels for quantification were randomised to reduce biases associated to labelling.

Briefly, plasma was fractionated using size exclusion chromatography under the following conditions: five columns serially connected as follows; 2 columns Shodex KW-804, 8.0 mm I.D.×300 mm, one column Shodex KW-802.5, 8.mm I.D.×300 mm 15 and 2 columns Shodex KW-804, operated at 45° C. and 1.5 mL/min under isocratic elution with 6M guanidine hydrochloride and 10% ethanol. Five SEC segments were collected in a peak-dependent fashion detected at 280 nm and then stored at −20° C. until further analysis.

The first four segments collected from the prefractionation of the plasma samples selected for this study and a master pool aliquot were dialysed and the protein content quantified. 120 μg of protein was reduced, alkylated and trypsin digested overnight (16 h). iTRAQ labelling was conducted during 2 hours and labelled peptides were dried in a speed vac at room temperature. Fractionation of labelled peptides was conducted using offline C4—HPLC. Peptides were analytically reconstituted and pooled together with 100 μL of 3% phase mobile B (99.92% acetonitrile and 0.08% ammonium hydroxide) and 97% phase A (99.92% water and 0.08% ammonium hydroxide). Pooled peptides were then centrifuged at 16000×g for 10 minutes. The pellet was stored at −20° C. and the supernatant was injected for separation using a Kromasil, C4 column 30 (3.5 μm, 2.1 mm×150 mm) operated at 35° C. and 0.3 mL/min. Offline fractions were collected in a peak-dependent fashion and detected at 215 nm.

Offline fractions containing contaminants, the early and late fractions, were pooled together with the pellet obtained in the previous step, and then peptides were cleaned 35 using C18-Aq solid phase extraction cartridges. Eluents were polled together and separated by C4 HPLC. Offline fractions were reconstituted in 31 μL of loading solution (2% acetonitrile and 1% formic acid). For the analytical separation the AcclaimPepMap RSLC, 75 μm×25 cm, nano Viper, C18, 2 μm particle column retrofitted to a PicoTip emitter (FS360-20-10-D-20-C7) was used for multistep gradient elution. Mobile phase was composed of 2% acetonitrile, 0.1% formic acid, and mobile phase (B) was composed of 99.9% acetonitrile, 0.1% formic acid. The gradient elution method at flow rate 300 nL/min gradually increased mobile phase B. Online analysis by mass spectrometry was performed using the high resolution nano-ESI-LTQ-Velos Pro Orbitrap-Elite mass spectrometer (Thermo Scientific).

Two separated analyses for HCD and CID fragmentation for each one of the collected fractions were performed. The mass spectrometer was set so that from each full MS scan the ten most intense ions with charge=+2 were selected for MS/MS. The normalised collision energy for MS2 was 35.0%. Full MS scans and MS/MS scans were acquired at a resolution of 30000 or 60000 for profile-mode and 15000 for centroid-mode, respectively, with a lock mass option enabled for the 445.120025 m/z ion. Xcalibur software was used for data acquisition. The LTQ FT-Orbitrap system was externally mass calibrated every 3-4 days using the positive ion calibration solution (Thermo Pierce, Rockford, IL, USA). Ion tuning was verified on a weekly basis as recommended by the manufacturer.

### Bioinformatic Analysis

Protein identifications were combined across plasma segments to give a grouped protein abundance for each plasma sample in the experiment in multi-consensus analysis. Plasma segment and multi-consensus analyses abundance data were then adjusted for experimental batch effects. Plasma segment data were analysed by linear modelling, and multi-consensus data were analysed by both linear modelling and whole genome network correlation analysis. Proteins identified by all three approaches are candidate diagnostic biomarkers.

### Validation Proteomics

Where assays existed for proteins that were significantly upregulated in pulmonary tuberculosis validation was performed by proximity extension assay using the cardiometabolic and inflammation 2 Olink® Explore panels. Dual antigen recognition is required by antibody binding in proximity which allows hybridisation and amplification of attached oligonucleotides, which are tagged with sequences specific to the sample and the antigen. Normalised protein expression is then calculated from the DNA sequencing readout.

The workflow is summarised in FIGS. 13 to 15. The data obtained is summarised in FIGS. 16 to 21.

1. A method of diagnosing tuberculosis in a subject, the method comprising:
a) providing a sample obtained from the subject;
b) determining the level of at least one protein in the sample, wherein the at least one protein is selected from the group consisting of Transcription termination factor 1, Fetuin-B, Heparin cofactor 2, Alpha-1-antichymotrypsin, Gamma-glutamyl hydrolase, Plastin-2, low affinity immunoglobulin gamma Fc region receptor III-B, and variants thereof;
c) comparing the level of the at least one protein from step b) with a reference level of the at least one protein; and
d) using the results from c) to diagnose or determine if the subject has tuberculosis,
wherein the reference level of the at least one protein is the level of the at least one protein that is present in an individual that does not have tuberculosis, and
wherein the subject is identified as having tuberculosis if the level of the at least one protein from step b) is higher than the reference level of the at least one protein.

2. The method of claim 1, wherein step b) comprises determining in the sample the level of Transcription termination factor 1 or a variant thereof and one or more proteins selected from Fetuin-B, Heparin cofactor 2, Alpha-1-antichymotrypsin, Gamma-glutamyl hydrolase, Plastin-2, low affinity immunoglobulin gamma Fc region receptor III-B, and variants thereof.

3. The method of claim 2, wherein step b) comprises determining in the sample the level of Transcription termination factor 1 or a variant thereof and Fetuin-B or a variant thereof.

4. The method of any one of the preceding claims, wherein step b) further comprises determining the level of at least one other protein biomarker of tuberculosis in the sample.

5. The method of claim 4, wherein the at least one other protein biomarker of tuberculosis is selected from Table 1.

6. The method of any one of the preceding claims, wherein step b) comprises determining the level of six proteins in the sample, wherein at least one protein is Transcription termination factor 1 or a variant thereof.

7. The method of any one of the preceding claims, wherein step b) further comprises determining the level of at least one protein in the sample selected from the group consisting of Adenosine deaminase 2, Monocyte differentiation antigen, Leucine-rich alpha-2-glycoprotein, L-selectin and variants thereof.

8. The method of any one of the preceding claims, wherein step b) comprises determining in the sample the level of Transcription termination factor 1 or a variant thereof, Fetuin-B or a variant thereof and at least one, at least two, at least three, at least four or at least five other protein biomarker(s) of tuberculosis selected from Table 1.

9. The method of any one of the preceding claims, wherein step b) comprises determining in the sample the level of Transcription termination factor 1 or a variant thereof, Fetuin-B or a variant thereof, Low affinity immunoglobulin gamma Fc region receptor III-B, or a variant thereof, and optionally one or more proteins selected from Heparin cofactor 2, Alpha-1-antichymotrypsin, Gamma-glutamyl hydrolase, Plastin-2, Adenosine deaminase 2, Monocyte differentiation antigen, Leucine-rich alpha-2-glycoprotein, L-selectin and variants thereof.

10. The method of any one of the preceding claims, wherein the subject is identified as having tuberculosis if the level of the protein from step b), or each of the proteins from step b) if more than one, is at least about 30% higher than the reference level of the protein.

11. The method of any one of the preceding claims, wherein the subject is human.

12. The method of any one of the preceding claims, wherein the tuberculosis is caused by Mycobacterium tuberculosis.

13. A method of treating tuberculosis in a subject, wherein the method comprises:
e) providing a sample obtained from the subject;
f) determining the level of at least one protein in the sample, wherein the at least one protein is selected from the group consisting of Transcription termination factor 1, Fetuin-B, Heparin cofactor 2, Alpha-1-antichymotrypsin, Gamma-glutamyl hydrolase, Plastin-2, low affinity immunoglobulin gamma Fc region receptor III-B, and variants thereof;
g) comparing the level of the at least one protein from step b) with a reference level of the at least one protein; and
h) administering a tuberculosis therapy to the subject if the level of the at least one protein from step b) is higher than the reference level of the at least one protein,
wherein the reference level of the at least one protein is the level of the at least one protein that is present in an individual that does not have tuberculosis.

14. A method of selecting a subject for treatment for tuberculosis comprising:
e) providing a sample obtained from the subject;
f) determining the level of at least one protein in the sample, wherein the at least one protein is selected from the group consisting of Transcription termination factor 1, Fetuin-B, Heparin cofactor 2, Alpha-1-antichymotrypsin, Gamma-glutamyl hydrolase, Plastin-2, low affinity immunoglobulin gamma Fc region receptor III-B, and variants thereof;
g) comparing the level of the at least one protein from step b) with a reference level of the at least one protein; and
h) selecting the subject for treatment if the level of the at least one protein from step b) is higher than the reference level of the at least one protein, wherein the reference level of the at least one protein is the level of the at least one protein that is present in an individual that does not have tuberculosis.

15. A kit comprising at least one agent for detecting the level of at least one protein selected from the group consisting of Transcription termination factor 1, Fetuin-B, Heparin cofactor 2, Alpha-1-antichymotrypsin, Gamma-glutamyl hydrolase, Plastin-2, low affinity immunoglobulin gamma Fc region receptor III-B, and variants thereof.
